(19) United States
(12) Patent Application Publication
(10) Pub. No.: US 2024/0173667 A1
(43) Pub. Date: May 30, 2024

(54) PLASMA FILTER FACILITY, ELECTRODE FACILITY AND METHOD FOR OPERATING A PLASMA FILTER FACILITY

(71) Applicant: Siemens Healthcare GmbH, Erlangen (DE)

(72) Inventors: Martin SEIFERT, Bayreuth (DE); Florian MEISE, Hausen (DE); Volker MODEL, Fuerth (DE); Lambert FEHER, Stutensee (DE); Philipp HOECHT, Lauf (DE)

(73) Assignee: Siemens Healthcare GmbH, Erlangen (DE)

(21) Appl. No.: 18/519,198

(22) Filed: Nov. 27, 2023

(30) Foreign Application Priority Data
Nov. 30, 2022 (EP) 22210434.1

(51) Int. Cl. B01D 53/32; A61L 9/20; B01D 46/00; B01D 53/00; B01D 53/04

(52) U.S. Cl. CPC B01D 53/32; A61L 9/20; B01D 46/0027; B01D 53/007; B01D 53/04; A61L 2209/14; B01D 2253/102; B01D 2259/804; B01D 2259/818; B01D 2279/65

(57) ABSTRACT

A plasma filter device includes at least one electrode device. The at least one electrode device having a first composite electrode and a second composite electrode in a planar manner. The first and second composite electrodes are arranged coplanar to one another on a main surface plane of the electrode device and are separated from one another by a discharge gap. Each of the first and second composite electrodes has a respective electrode sheet that, at least on a boundary surface of the electrode sheet to the discharge gap, has a respective dielectric coating. The plasma filter device further includes a power source configured to provide an AC voltage to the electrode device. The AC voltage is parameterized to instigate formation of a plasma through a dielectric barrier discharge in the discharge gap.

### CROSS-REFERENCE TO RELATED APPLICATION (S)

The present application claims priority under 35 U.S.C. § 119 to European Patent Application No. 22210434.1, filed Nov. 30, 2022, the entire contents of which is incorporated herein by reference.

### FIELD

One or more example embodiments of the present invention relate to a plasma filter facility (or device) having at least one electrode facility (or device), to an electrode facility (or device) and also to a method for operating a plasma filter facility (or device).

### BACKGROUND

Independent of the grammatical term usage, individuals with male, female or other gender identities are included within the term.

Even before the Coronavirus pandemic the prevention of the transmission of pathogens by aerosols was a challenge for hospital hygiene. Previously the reduction of germs and particles has only been carried out in specific rooms such as operating theatre, laboratory, and isolation unit. Due to the pandemic the demand for pathogen-free ventilation in combination with ventilation and air-conditioning systems has become increasingly more important, however. Currently various filter solutions are available on the market based on plasma, UVC radiation and their combinations with additional filters, such as for example HEPA filters for the conditioning of air in closed environments such a buildings, automobiles, aircraft or trains.

Due to the very specific requirement of the various possible applications, solutions available on the market have disadvantages with regard to the flow mechanics of the gas to be filtered or the size of the filter apparatus. Moreover existing solutions can give rise to high costs, which can arise from filter units to be provided and from a regular exchange and a disposal of additional filter elements such as HEPA.

Thus an effective as well as efficient incorporation into and optimization of filter solutions currently employed is only possible under some conditions or is impossible.

Known filter apparatuses according to the prior art include mechanical air filters for example.

Currently the mechanical filtration of ambient air is of great importance for keeping the ambient air clean and for sterilizing it, in particular in hospitals and other medical facilities. Modern building air cleaning systems have multi-stage filter arrangements, which consist of prefiltering of dust for example and high-efficiency filtering for microbial contamination for example. In operating applications these are combined with mechanical apparatuses in order to create a laminar (turbulence-free) flow of air over the patient. In today's operation theater installations (so-called ventilation and air conditioning systems) fresh air is supplied that, above all in winter, has to be heated up to the normal room temperature (20-22 degrees) using large amounts of energy.

With a degree of separation of at least 99.95% highly efficient HEPA filters effectively hold back bacteria and viruses as well as particle contamination. SARS-COV-2 viruses also occur in mesoscale aerosols, which can pass through these filters. In particular these fine aerosols have a long dwell time in the air.

For buildings it was proposed in the pandemic in the year 2021 that the ventilation be improved with HEPA filters. This resulted in large and costly expansion of systems. This also leads to a significant worsening of the energy balance by compensation for pressure losses, as well as, through an increase of the flow speed, in increased emission of noise.

For aircraft central filter systems with HEPA units are used in order to clean the air. The size, the weight and the energy consumption influence the overall performance of an aircraft through this ventilation system. Automobile/bus/train compartments only consist of central air conditioning chambers with simple filter technology that is not suitable for making a decontamination of the air possible.

As well as mechanical filter apparatuses, filter apparatuses are also employed that make possible UVC or plasma-based methods for decontamination of air.

Widely-used ozone generators, ionizers or plasma systems consist of comprehensive interaction chambers. In order to decontaminate ambient air with UV lamps for example, tunnel-like systems with slow to gentle air flows are used. Only by this can high decontamination rates be obtained. The common factor with all these filter apparatuses is therefore that they are used as central units with an expanded cube-shaped or spatially longitudinal design.

In recent times mobile surface decontamination devices or stationary UVC lamps have been employed for surface decontamination. A disadvantage of these solutions available on the market is in their implementation in for example medical imaging devices, due to the requirements for example in relation to installation space, EMC or the air flow conditions.

Solutions available on the market are based on electromagnetic discharges and a comparatively slow disinfection effect by radicals.

More advanced solutions therefore additionally combine UVC-based methods with the plasma generated. With UVC-based methods the power of the dose necessary for an effective and efficient decontamination of the air is heavily dependent on the wavelength of the UVC source used and the time that the ambient air stays in the corresponding filter unit. Experiences from surface decontamination by UVC radiation have shown that in some cases long irradiation times depending on the distance and the power of the dose are necessary in order to guarantee and high level of germ elimination.

In relation to medical devices, a CT machine with a self-cleaning function is known from CN203524686U. A sterilization unit for the CT machine based on UVC, plasma or ozone is disclosed here.

### SUMMARY

An object of one or more example embodiments of the present invention is to provide a filter facility (also referred to as a filter device) that makes possible a more efficient decontamination of air.

At least this object is achieved by the respective subject matter of the independent claims. Advantageous developments and preferred forms of embodiment are the subject matter of the dependent claims.

A first aspect of an embodiment of the present invention relates to a plasma filter facility (also referred to as a filter device), which has at least one electrode facility (also referred to as an electrode device). The plasma filter facility involves a filter facility (also referred to as a filter device) for filtering a gas, for example for filtering of air, wherein a plasma is generated for filtering the gas. UV radiation is emitted by the generated plasma, by which for example aerosols or germs to be found in the gas can be inactivated or particles can be deactivated.

There is provision for the electrode facility to have a first composite electrode embodied in a planar manner and a second composite electrode embodied in a planar manner. The composite electrodes can for example comprise uniform, symmetrical elements, which can be arranged in an antisymmetric electromagnetic potential contour. In other words the electrode facility comprises the first composite electrode and the second composite electrode. The composite electrodes of the electrode facility can in particular be configured as surface elements. There is provision for the composite electrodes of the electrode facility to be arranged coplanar to one another in a main surface plane of the electrode facility and to be separated from one another spatially by a discharge gap. In other words the composite electrodes of the electrode facility are located in the main surface plane of the electrode facility. Located between the composite electrodes of the electrode facility is a discharge gap. The discharge gap involves a gap of the electrode facility formed by the composite electrodes for the passage of gas to be filtered. There is provision for each of the composite electrodes to have a respective electrode sheet that, at least on a boundary surface of the respective electrode sheet to the discharge gap, has a respective dielectric coating. In other words the electrodes are provided as composites, which include the respective electrode sheet and a dielectric coating located on the respective electrode sheet. The dielectric coating is applied to the electrode sheet at least on a boundary surface of the respective composite electrode adjoining the discharge gap.

The plasma filter facility has a power source, which is configured to provide an AC voltage to the electrode facility, wherein the AC voltage is parameterized to instigate a formation of plasma through a dielectric barrier discharge in the discharge gap. In other words there is provision for the plasma filter facility to have the power source. The power source is intended for providing the AC voltage to the electrode facility in order to instigate the formation of the plasmas in the discharge gap.

The plasma filter facility is configured to guide a gas along a main direction of flow, which is aligned in parallel to a normal of the main surface plane of the electrode facility, through the discharge gap. In other words the plasma filter facility has flow guidance elements, for example tube elements, which are configured to influence or to define a main direction of flow of the gas in such a way that the gas is guided through the discharge gap in parallel to the normals of the main surface plane.

The main direction of flow in this case runs in parallel to the normals of the main surface plane of the electrode facility. In other words the main direction of flow runs at right angles to the electrode facility. The gas thus flows through the electrode facility at right angles through the discharge gap. Due to the plasma generated in the discharge gap by the dielectric discharge, a decontamination of the gas takes place in the area of the discharge gap. The decontamination can be carried out by UVC rays that can be emitted by the plasma and/or by ozone that can form in the discharge gap when the gas involved is air.

An advantage produced by an embodiment of the present invention is that a particle trap is provided by the discharge gap in which particles stay longer than molecules of the gas itself. Due to the particles remaining longer in the area of the discharge gap, these are subjected over a longer period of time to plasma effects such as radicals and UVC radiation, whereby a probability of an inactivation of the particle increases. For example with a UVC power of 100 W/m2 and a dwell time of the particles of just 1 second, a dose power of 100 J/m2 is produced. Corona viruses are already 90% inactivated as from 37 J/m2.

One or more example embodiments of the present invention also comprise developments, through which further advantages are produced.

One development of an embodiment of the present invention makes provision for each of the composite electrodes to have a comb structure, which has electrode fingers. In other words the composite electrodes comprise a respective comb structure made of electrode fingers. The comb structure can be an arrangement of electrode fingers aligned in parallel to one another, which can be arranged next to one another spaced apart by a predetermined distance. The comb structures of the composite electrodes of the electrode facility are arranged engaging in and separated from one another by the discharge gap. In other words the comb structures of the composite electrodes of the electrode facility are arranged in relation to one another in such a way that the electrode finger of the other composite electrode is arranged between two electrode fingers of one of the composite electrodes. The distances between the electrode fingers of the comb structure of the respective composite electrode are arranged in such a way in this case that the distance is greater than a width of the electrode finger of the other electrode. The electrode fingers of the composite electrodes are not in contact with one another but are separated spatially from one another by the discharge gap. The electrode sheets of the comb structure can feature metallic or generally electrically highly conductive materials. Due to the engaging comb structures of the composite electrodes of the electrode facility the discharge gap in the electrode facility has a serpentine structure. The advantage produced by the development is that, through the serpentine course of the discharge gap, on the one hand a more marked effect of the discharge gap as a particle trap is produced than with other courses, on the other hand an optimal antisymmetric electromagnetic potential contour with perfectly linearly polarized electrical fields is followed.

One development of an embodiment of the present invention makes provision for the electrode sheet of each of the composite electrodes to feature aluminum and/or an aluminum alloy. In other words a material of the electrode sheet features aluminum or the aluminum alloy.

One further solution of an embodiment of the present invention makes provision for the electrode sheet of each of the composite electrodes to feature non-corrosive steel, preferably stainless steel. In other words the electrode sheet has a material that comprises non-corrosive stainless steel. This can involve steel that features chromium for example.

One development of an embodiment of the present invention makes provision for the dielectric coating of each of the composite electrodes to feature one or more polymers. In other words the dielectric coating involves a material that features one or more polymers, in particular electrically insulating polymer substances.

One development of an embodiment of the present invention makes provision for the dielectric coating of each of the composite electrodes to feature on or more fluoroplastics. In other words the dielectric coating features a material that comprises fluoropolymers. The advantage produced by this is that the dielectric coating features polymers with a relatively high insulation capability.

One development of an embodiment of the present invention makes provision for the dielectric coating of each of the composite electrodes to feature polyvinylidene fluoride (PVDF). In other words the dielectric coating features a material that comprises polyvinylidene fluoride (PVDF).

One development of an embodiment of the present invention makes provision for the dielectric coating of each of the composite electrodes to feature polytetrafluoroethylene (PTFE). In other words the dielectric coating features a material that comprises polytetrafluoroethylene (PTFE). The use of polytetrafluoroethylene (PTFE) has the advantage that it involves a substance with a relatively high corrosion and temperature resistance.

One development of an embodiment of the present invention makes provision for the dielectric coating of each of the composite electrodes to feature graphite fluoride. In other words the dielectric coating features a material that comprises graphite fluoride.

One development of an embodiment of the present invention makes provision for dielectric coating of each of the composite electrodes to feature one or more ceramics. In other words the dielectric coating features a material that comprises one or more ceramics. It can in particular involve ceramics of the electroceramics group. Possible ceramics can for example comprise titanate, in particular barium titanate ceramics and/or lead titanate zirconate ceramics. The use of ceramics has the advantage that substances having a relatively high dielectric strength are involved.

One development of an embodiment of the present invention makes provision for the dielectric coating of each of the composite electrodes to feature barium titanate. In other words the dielectric coating features a material that comprises barium titanate. The use of barium titanate has the advantage that a substance having a relatively high dielectricity constant is involved.

One development of an embodiment of the present invention makes provision for the dielectric coating of each of the composite electrodes to feature kaolinite. In other words the dielectric coating features a material that comprises kaolinite. The use of kaolinite has the advantage that a substance having a relatively high dielectricity constant is involved.

One development of an embodiment of the present invention makes provision for the dielectric coating of each of the composite electrodes to feature a blend, comprising predetermined proportions of kaolinite, aluminum oxide, titanium oxide, chromium oxide, barium titanate and/or other ceramic powders. In other words the dielectric coating of each of the composite electrodes features specific proportions of kaolinite, aluminum oxide, titanium oxide, chromium oxide or barium titanate or other ceramic powders. In other words the dielectric coating features a material that comprise a ceramic blend. The use of the blend has the advantage that a substance having a specific optimally adapted dielectricity constant is involved. A blend can also be referred to as a mixture.

One development of an embodiment of the present invention makes provision for the plasma filter facility to feature at least two of the electrode facilities. In other words the plasma filter facility features two or more than two of the electrode facilities. The plasma filter facility has a holder facility (also referred to as a holder device) as a housing, which is configured to arrange the at least two electrode facilities in a predefined electrode arrangement, wherein the at least two of the electrode facilities in the predefined electrode arrangement are aligned in parallel to one another and arranged behind one another along the main direction of flow. In the predefined electrode arrangement neighboring electrode facilities are arranged separated by a predefined distance from one another in pairs. In other words the plasma filter facility is configured via the holder facility to provide the arrangement of the electrode facilities in the predefined electrode arrangement. There is provision for the electrode facilities to be aligned by the holder facility in parallel to one another. There is moreover provision for the electrode facilities to be arranged by the holder facility in such a way that these are arranged behind one another along the main direction of flow. The electrode facilities can for example be shifted in relation to one another along the main direction of flow. The holder facility is configured to arrange the at least two electrode facilities in the electrode arrangement such that these are separated from one another in pairs by a predefined distance. The holder facility can for example have a separation frame, which is arranged between two of the respective electrode facilities and can predetermine the predefined distance between the electrode facilities. The advantage produced by the development is that the gas can be guided along the main direction of flow through a number of electrode facilities arranged behind one another, whereby a number of deactivation steps are performed while gas is flowing through the filter facility.

One development of an embodiment of the present invention makes provision for neighboring electrode facilities of the at least two electrode facilities to be aligned rotated at 90° to one another about the main direction of flow. In other words there is provision for electrode facilities arranged behind one another to be rotated through 90°. There can be provision for example for the electrode facilities to have the same pattern predetermined by the discharge gap in the main surface plane, wherein the pattern is rotated through 90° between neighboring electrode facilities. This enables the patterns of the electrode facilities along the main direction of flow to form a cross structure. The advantage produced by the development is that a dwell time of the aerosols and preferably of the floating aerosols in neighboring electrode facilities can be increased.

One development of an embodiment of the present invention makes provision for the plasma filter facility to have at least one dust filter. The at least one dust filter is arranged in a main direction of flow before the electrode facility. In other words the plasma filter facility is configured to first guide the gas through the dust filter along the main direction of flow, before the gas is guided through the electrode facility. The dust filter can for example involve a mechanical dust filter, which has a mesh through which the gas is routed for filtering. An electrostatic dust filter can also be involved. The advantage produced by the development is that particles or dirt or larger particles can be trapped by the dust filter, so that a blockage of the electrode facility due to dust or impurities can be delayed or prevented.

One development of an embodiment of the present invention makes provision for the plasma filter facility to have at least one active carbon filter. There is provision for the at least one active carbon filter to be arranged in a main direction of flow after the electrode facility. In other words the plasma filter facility is configured to guide the gas along the main direction of flow after it has flowed through the electrode facility, through the at least one active carbon filter. The advantage produced by the development is that specific, undesired short-lived radicals (plasma-specific transient charge-carrying ions/molecules/atoms), which can have been formed in the plasma can be filtered out catalytically by neutral recombination by the active carbon filter. For example, due to the formation of plasma in the discharge gap there can be resulting formation of ozone. An output of ozone is however undesired in specific situations. Due to the provision of the active carbon filter the corresponding ozone is recombined, so that the ozone part of the gas emerging from the plasma filter facility can be reduced catalytically to an amount tolerable for clean air.

A second aspect of embodiments of the present invention relates to an electrode facility for a plasma filter facility. There is provision for the electrode facility to be arranged in a plasma filter facility for decontamination of a gas.

There is provision for the electrode facility to have a first composite electrode embodied in a planar manner and a second composite electrode embodied in a planar manner. In other words the electrode facility comprises the first composite electrode and the second composite electrode. The composite electrodes of the electrode facility can be configured in particular as surface elements. There is provision for the composite electrodes of the electrode facility to be arranged in a coplanar manner in relation to each other in a main surface plane of the electrode facility and to be separated spatially from one another by a discharge gap. In other words the composite electrodes of the electrode facility are located in the main surface plane of the electrode facility. Located between the composite electrodes of the electrode facility is a discharge gap. The discharge gap involves a gap of the electrode facility formed by the composite electrodes for the passage of the gas to be filtered. There is provision for each of the composite electrodes to have a respective electrode sheet that, at least on a boundary surface of the respective electrode sheet to the discharge gap, has a respective dielectric coating. In other words the electrodes are provided as composites, which comprise the respective electrode sheet and a dielectric coating to be found on the respective electrode sheet. The dielectric coating is applied at least to one of the respective composite electrodes to the boundary surface on the electrode sheet adjoining the discharge gap.

Further forms of embodiment of the inventive electrode facility follow from the different forms of embodiment of the inventive plasma filter facility.

A third aspect of embodiments of the present invention relates to a method for operating a plasma filter facility.

The plasma filter facility has at least one electrode facility, wherein the electrode facility has a first composite electrode embodied in a planar manner and a second composite electrode embodied in a planar manner. In other words the electrode facility comprises the first composite electrode and the second composite electrode. The composite electrodes of the electrode facility can in particular be configured as surface elements. There is provision for the composite electrodes of the electrode facility to be arranged in a coplanar manner in relation to one another in the main surface plane of the electrode facility and to be spatially separated from one another by a discharge gap. In other words the composite electrodes of the electrode facility are located in the main surface plane of the electrode facility. A discharge gap is to be found between the composite electrodes of the electrode facility. The discharge gap involves a gap of the electrode facility formed by the composite electrodes for the passage of the gas to be filtered. There is provision for each of the composite electrodes to have a respective electrode sheet that, at least on a boundary surface of the respective electrode sheet to the discharge gap, has a respective dielectric coating. In other words the electrodes are provided as composites, which comprise the respective electrode sheet and a dielectric coating to be found on the respective electrode sheet. The dielectric coating is applied to the electrode sheet at least on a boundary surface of the respective composite electrode adjoining the discharge gap.

In the method there is provision for a power source of the plasma filter facility for an AC voltage, preferably in the increased AC voltage range (100-1000 Hz) or in the lower high frequency range (Kilohertz) and also in the high and highest frequency range (1 GHZ-100 GHz) to be provided to the electrode facility, whereby a plasma is formed due to a dielectric barrier discharge instigated in the discharge gap. Through the plasma filter facility a gas is guided along a main direction of flow, which is aligned in parallel to a normal of the main surface plane of the electrode facility, through the discharge gap. Due to the plasma the result is a decontamination of the gas, wherein germs are killed for example.

Further forms of embodiment of the inventive method follow from the different forms of embodiment of the inventive plasma filter facility and also of the electrode facility.

Further features of embodiments of the present invention emerge from the claims, the figures and the description of the figures. The features and combinations of features given here in the description as well as the features and combinations of features given below in the description of the figures can be included not just in the respective combination specified, but also in other combinations of the invention. In particular versions and combinations of features of embodiments of the present invention can be included that do not have all features of an originally formulated claim. Above and beyond this versions and combinations of features can be included in embodiments of the present invention that go beyond the combinations of features set out in the references of the claims or deviate from said references.

### BRIEF DESCRIPTION OF THE DRAWINGS

The present invention will be explained in greater detail below with the aid of concrete exemplary embodiments and associated schematic diagrams. In the figures elements that are the same or have the same function can be provided with the same reference characters. The description of the same elements or elements with the same functions may possibly not necessarily be repeated with regard to different figures. In the figures:

FIG. 1 shows a schematic diagram of an electrode facility for a plasma filter facility;

FIG. 2 shows a schematic diagram of an electrode facility;

FIG. 3 shows a schematic diagram of a way in which the electrical electrode facilities function;

FIG. 4 shows a schematic diagram of a plasma filter facility;

FIG. 5 shows a schematic diagram of a plasma filter facility;

FIG. 6 shows a schematic diagram of a simulation of a volume air flow through the plasma filter facility;

FIG. 7 shows a schematic diagram of a transmission of aerosols through an electrode facility;

FIG. 8 shows a schematic diagram of transmission of aerosols through two electrode facilities; and

FIG. 9 shows a schematic diagram of possible arrangements of the electrode facilities.

### DETAILED DESCRIPTION

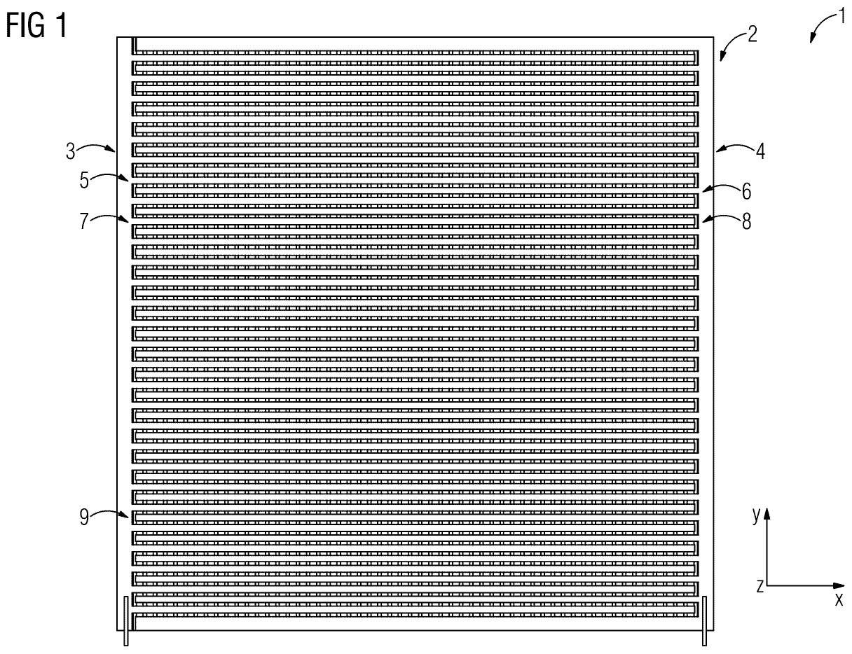

FIG. 1 shows a schematic diagram of an electrode facility for a plasma filter facility.

The electrode facility 2 of the plasma filter facility 1 can have a first composite electrode 3 and a second composite electrode 4. The first composite electrode 3 and the second composite electrode 4 can lie in a main surface plane 13 of the electrode facility 2 and can be planar. The first composite electrode 3 and the second composite electrode 4 can for example be arranged in a coplanar manner in relation to one another. The composite electrodes 3, 4 can have been made from an original sheet of metal, which can be separated by a provision of a discharge gap 9 in the original sheet of metal into a first electrode sheet 5 and a second electrode sheet 6, wherein the first electrode sheet 5 and the second electrode sheet 6 can be separated from one another by the discharge gap 9. The two composite electrodes 3, 4 can have a respective electrode sheet 5, 6, which can be coated with a respective dielectric coating 7, 8, whereby the respective electrodes can involve composites. The electrode sheet 6, 6 of respective composite electrode 3, 4 can for example have aluminum, an aluminum alloy and/or stainless steel as a material. The dielectric coating 7, 8 of the respective composite electrode 3, 4, at least on a boundary surface of the respective composite electrode 3, 4 to the discharge gap 9, can be applied to the respective electrode sheet 5, 6. The task of the dielectric coating 7, 8 can consist of making possible a dielectric discharge in the discharge gap 9 when an appropriately parameterized electrical AC voltage is applied to the electrode facility 2. The dielectric coating 7, 8 can have one or more polymers as its material. The possible polymers can for example feature fluoroplastics, in particular polyvinylidene difluoride and/or polytetrafluorethylene. A graphite fluoride can be mixed with the at least one polymer. The dielectric coating 7, 8 can also have one or more ceramics as its material, in particular barium titanate.

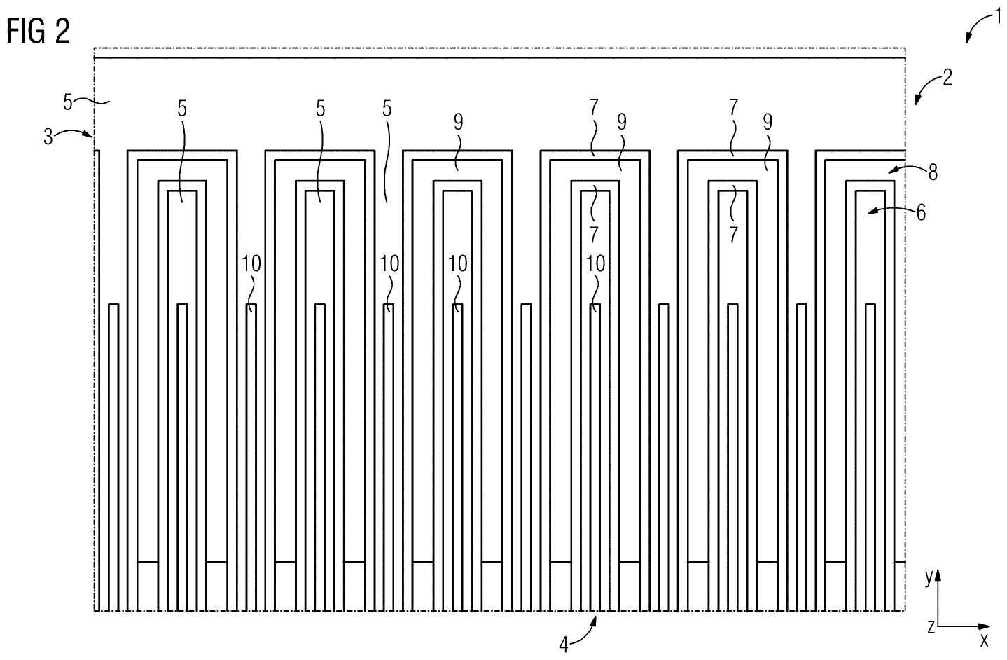

FIG. 2 shows a schematic diagram of an electrode facility.

FIG. 2 shows comb structures of the composite electrodes 3, 4 of the electrode facility 2. The first composite electrode 3 of the electrode facility 2 can have a comb structure that can comprise electrode fingers, which can be arranged in parallel to and spaced apart 15 from one another. Correspondingly the second composite electrode 4 of the electrode facility 2 can also have a comb structure with electrode fingers, which can engage in spaces between the composite electrodes 3, 4. The electrode fingers of the respective composite electrodes 3, 4 can be spaced apart 15 from one another by the discharge gap 9. The surfaces of the electrode sheets 5, 6 can have the dielectric coatings 7, 8 at least in the area of the discharge gap 9. The comb structures can also comprise dielectric gaps 10 made of dielectrics, which can be applied to the electrode sheets 5, 6 along respective center lines of the electrode fingers.

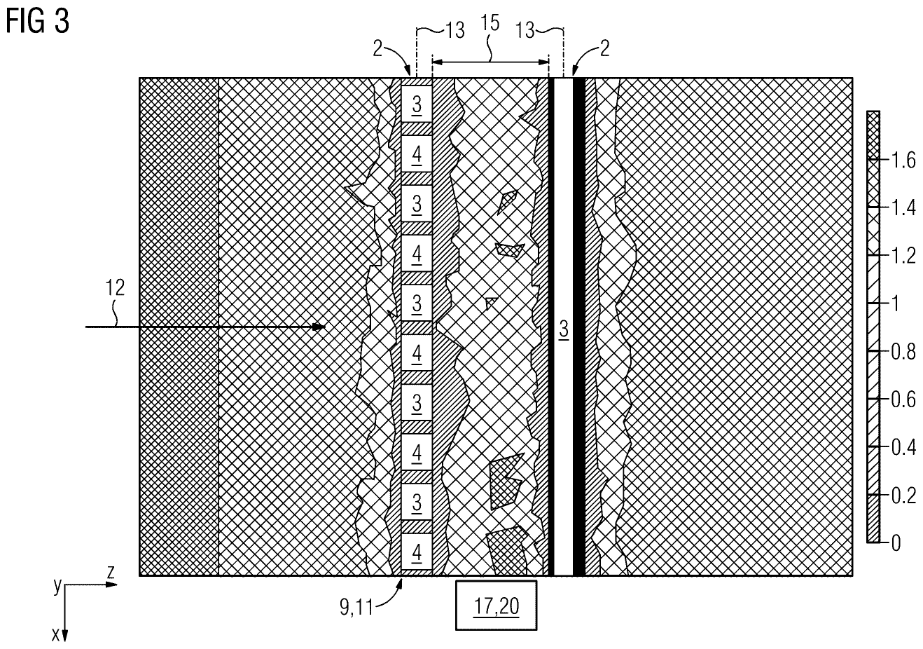

FIG. 3 shows a schematic diagram of a way in which the electrical electrode facilities function.

The figure shows two of the electrode facilities 2, which can be arranged behind one another in relation to a main direction of flow 12 of the gas through the plasma filter facility 1. The plasma filter facility 1 can be provided to convey the gas to be decontaminated along the main direction of flow 12 of the gas through the first electrode facility 2 and the second electrode facility 2, wherein the main direction of flow 12 can be aligned in parallel to a normal of the main surface planes 13 of the electrode facilities 2. A distribution of amounts of velocity in m/s of particles is shown, which can be found in the gas conveyed through. Due to the right-angled alignment of the main direction of flow 12 in relation to the electrode facility 2, the gas is guided through the discharge gaps 9 of the electrode facilities 2. Through a power source 22 not shown an AC voltage can be provided to the electrode facilities 2, which can lead to a formation of a plasma 11 in the discharge gaps 9 due to a dielectric discharge. The plasma 11 can emit UV radiation, which can deactivate aerosols in the gas. The deactivation can comprise bringing about a specific chemical reaction in an aerosol, through which for example germs can be killed. The electrode facilities 2 can be arranged by a facility 20 in a predetermined electrode arrangement and separated from one another by a distance 15. In the mapping of the velocity amounts it can be seen that the velocity has a minimum around the two electrode facilities 2. This is attributable to the fact that the serpentine form of the discharge gap 9 described in FIG. 2 leads to the discharge gap 9 functioning as a particle trap, whereby particles of the gas stay for a longer period of time in the respective discharge gap 9. Due to the greater time that the particles stay in the discharge gap 9, a particle is subjected over a longer period of time to the plasma 11 and thus to the ultraviolet radiation.

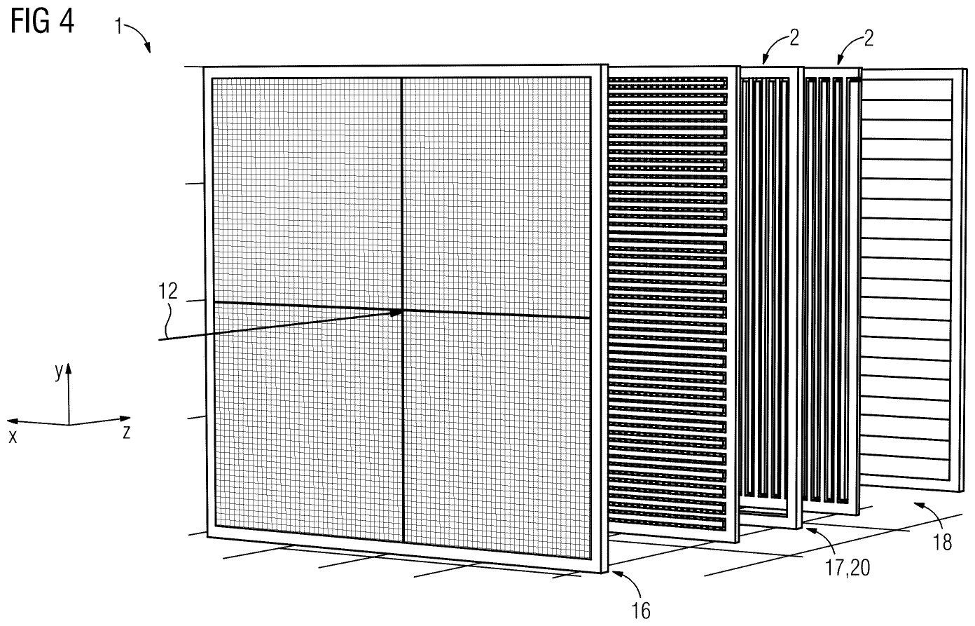

FIG. 4 shows a schematic diagram of a plasma filter facility.

The plasma filter facility 1 can have a dust filter 16, which can be arranged in relation to a main direction of flow 12 before the electrode facilities 2. The dust filter 16 can be provided for filtering out dust particles or larger aerosols from the gas guided through the plasma filter facility 1 before it passes through the electrode facility 2 in order to prevent a blockage and prevent and/or slow down a contamination of the electrode facility 2. A first electrode facility 2 can be arranged behind the dust filter 16. The electrode facility 2 can have the described serpentine structure, wherein the electrode fingers can be aligned along the X direction. The further electrode facility 2 can be arranged behind the first electrode facility 2, which can be arranged separated by a predetermined distance 15 by a separation 17 from the first electrode facility 2. The second electrode facility 2 can be rotated by 90° in relation to a normal running in the z direction. In this alignment the electrode fingers can for example be aligned in parallel to the y direction. The rotated alignment of the subsequent electrode facility 2 compared to the first electrode facility 2 enables a time that the particles stay in discharge gaps 9 of the plasma filter facility 1 to be lengthened compared to plasma filter facilities 1 with electrode facilities 2 not rotated in relation to one another. An active carbon filter 18, through which the gas flows can be arranged beyond the second electrode facility 2. The active carbon filter 18 can be provided to filter out ozone molecules, which can form in the plasma 11, from the gas.

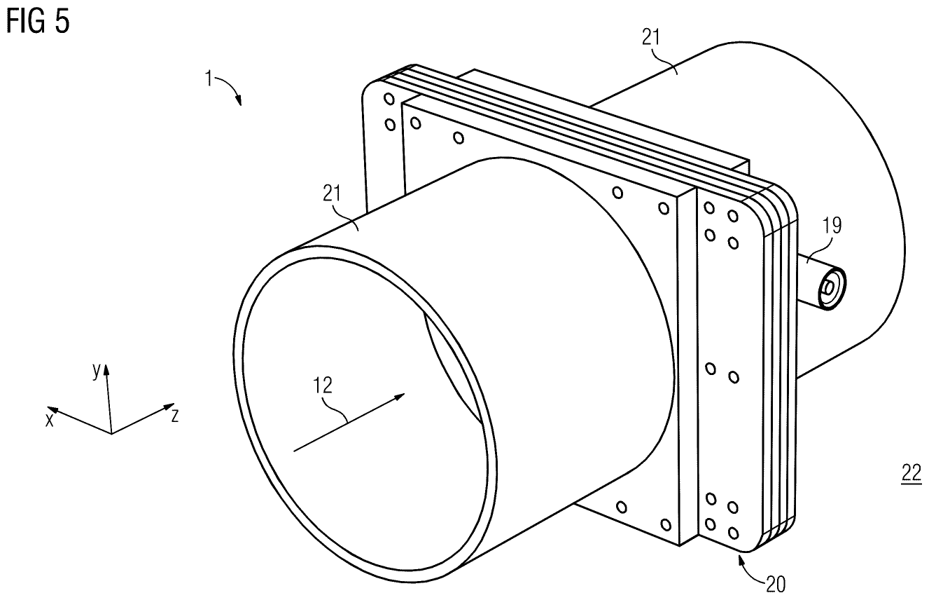

FIG. 5 shows a schematic diagram of a filter facility.

The plasma filter facility 1 can have a holder facility 20, which can be provided to arrange the electrode facilities 2, the dust filter 16 and the active carbon filter 18 in predetermined locations. A power supply point 19 can be arranged on the holder facility, which can be provided to supply the electrode facilities 2 with the AC voltage of the power source 22. In order achieve a conveyance of the gas along the main direction of flow 12, one or two tubes 21 or in general guide elements can be arranged on the plasma filter facility 1, which can be arranged via a flange on the holder facility 20.

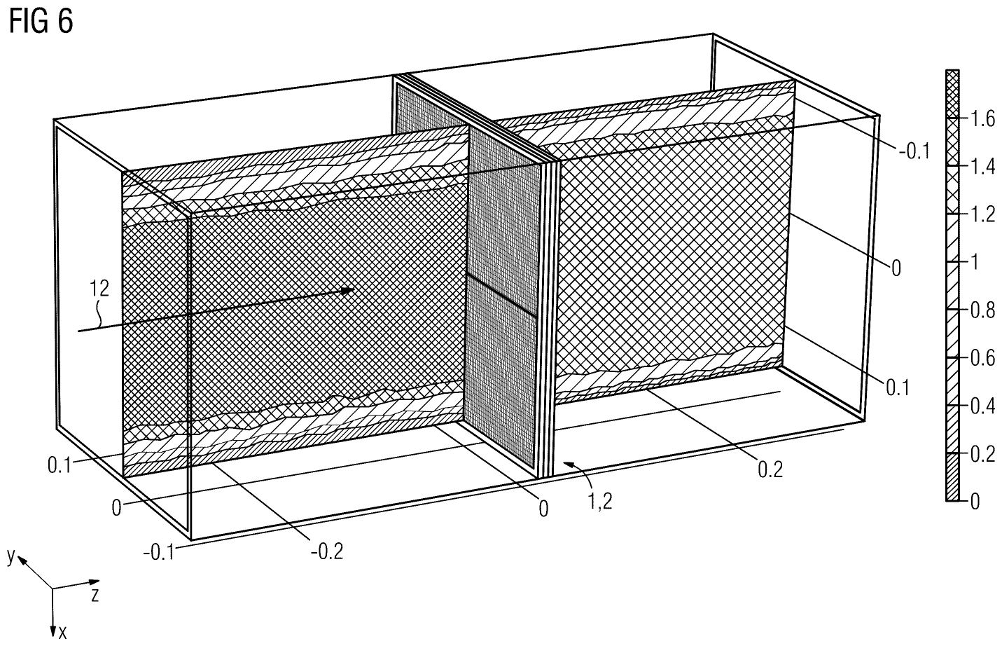

FIG. 6 shows a schematic diagram of a simulation of an air volume flow through a plasma filter facility.

What is shown is a simulation or the air volume flow through the present plasma filter unit in m/s.

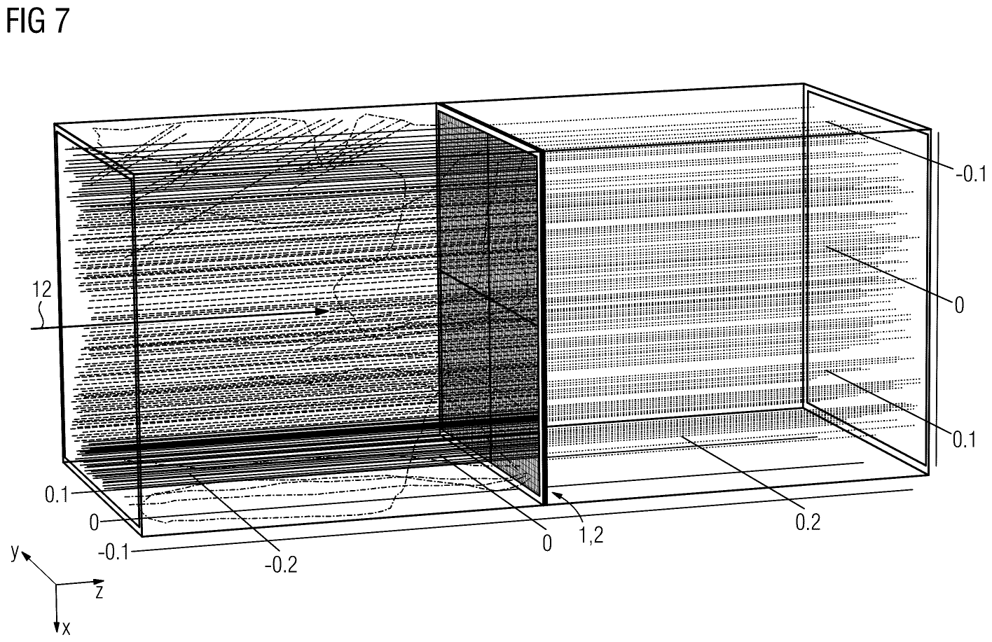

FIG. 7 shows a schematic diagram of an aerosol transmission through an electrode facility.

The figure FIG. 7 shows a schematic diagram of an aerosol transmission or aerosols of a size of 0.3 μm for a time of 600 seconds through an electrode facility 2. The simulation shows that the aerosols of the size of 0.3 μm stay for a relatively long time in the serpentine discharge gaps 9. What the figure shows is the velocity of the aerosols in m/s.

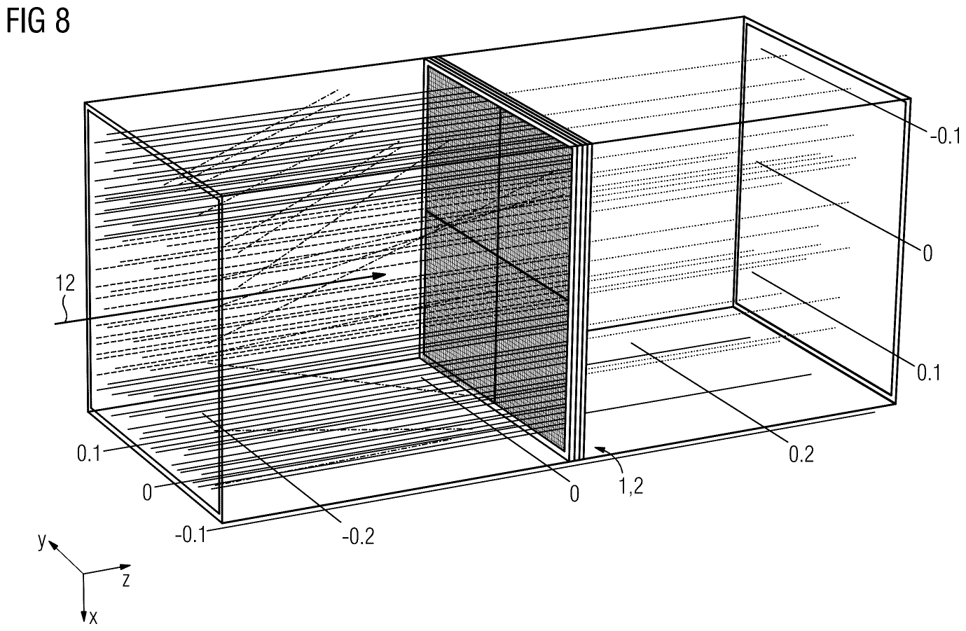

FIG. 8 shows a schematic diagram of an aerosol transmission through two electrode facilities.

The figure shows a schematic diagram of an aerosol transmission of aerosols of a size of 0.3 μm for 600 seconds through two of the electrode facilities 2. What is shown is the velocity of the aerosols in m/s. The two electrode facilities 2 are arranged behind one another and rotated by 90° in relation to one another. It can be seen that the particles stay longer in the plasma filter facility 1 that comprises two electrode facilities 2 compared to how long they stay in the plasma filter facility 1 that has one electrode facility 2. This can be seen by comparing FIG. 7 with FIG. 8.

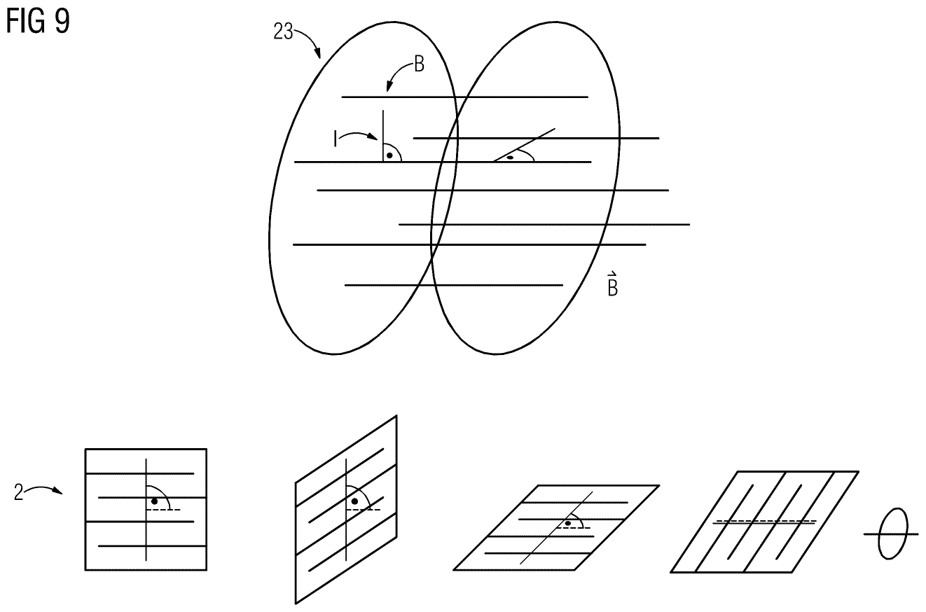

FIG. 9 shows a schematic diagram of possible arrangements of the electrode facilities 2.

The electrode facilities 2 are characterized by their preferred anisotropic direction. FIG. 9 shows the possibility of aligning the electrode facilities 2 so that a complete suppression of any influencing of the plasma 11 can be produced. The diagram shows Helmholtz coils 23 of an MRT apparatus with the B-field orientation B and orientations of electrode facilities 2 with plasma flow orientations I.

Technical features of embodiments of the present invention and advantages obtained thereby through the use of composite electrodes 3, 4 with defined design and arrangement. The arrangement will be referred to below as the plasma filter facility 1.

Embodiments of the present invention encompass at least two central ideas. The first central idea relates to a generation of a plasma 11 in order to generate high photon densities in the UVC wavelength range. The second central idea relates to a provision of a new electrode and filter design, through which a longer dwell time of specific aerosol particle fractions from a laminar gas volume flow in the plasma filter facility 1 is enforced.

The information below is intended to describe the hallmarks of the layout of the plasma filter facility 1 for gas decontamination in greater detail and to explain the two central ideas more precisely.

The first central idea concerns the generation of the plasma 11 using a defined electrode design. This central idea comprises a combination of the two metallic electrode sheets 5, 6, through which a serpentine structure of the electrode facility 2 is provided. The two electrode sheets 5, 6 of the electrode facility 2 can be manufactured by commercial and low-cost manufacturing methods according to the prior art, for example by punching and/or cutting an original sheet of metal. The original sheet of metal in this case can be worked so the serpentine course of the discharge gap 9 is provided in the original sheet of metal. Through the discharge gap 9 the original sheet of metal can be divided into the two electrode sheets 5, 6 of the electrode facility 2.

A dielectric coating 7, 8 consisting of a dielectric with a defined permittivity & in the range of 5-50 can be applied to the two electrode sheets 5, 6 of the electrode facility 2, whereby the composite electrodes 3, 4 can be provided. The two electrode sheets 5, 6 can preferably comprise Al, Al alloys or non-corrosive steels.

The dielectric of the dielectric coatings 7, 8 can for example feature polymers, for example PVDF, PTFE and other fluoro-related polarized polymers with a very high proportion of graphite fluoride C—F with a bonding energy of 489 KJ/mol. The dielectric of the dielectric coatings 7, 8 can also feature ceramics, for example barium titanate with a permittivity of 50.

A boundary condition of the simulation is a ceramic coating 7, 8 with an increased dielectric number of 12, in order to show the characteristic difference from a polymer-related dielectric coating 7, 8 with Epsilon=7. The thickness of the polymer-related dielectric coating 7, 8, due to the technical requirements of the injection molding process for application of the at least one polymer, is higher compared to the thickness of a ceramic dielectric coating 7, 8. In accordance with experience the thickness of the polymer-related dielectric coating 7, 8 can amount to 0.5 mm for example. The ceramic dielectric coating 7, 8 can have a thickness of 0.2 mm for example. The ceramic dielectric coating 7, 8 can be applied for example via a chemical gas phase deposition of the dielectric onto the respective electrode sheet.

Analysis shows that an enlargement of the air gap within specific areas advantageously increases the deposited power in plasma 11.

The plasma filter facility 1 can have electrode facilities 2 made of composite electrodes 3, 4, and also planar dust filter 16 and active carbon filter 18, as shown in FIG. The plasma filter facility 1 can have a very small size compared to known plasma filter facilities 1. The holder apparatus can have frames and enclosures that can comprise polymer materials.

For decontamination of the gas there is provision for generation of photons in the UVC wavelength range. The plasma filter facility 1 is configured to generate the photons in the air gap between the composite electrodes 3, 4 of the electrode facility 2 by a plasma 11. There is provision for the plasma 11 to be generated via a dielectric barrier discharge in the discharge gap 9.

The gas flowing through the plasma filter facility 1 becomes electrically conductive via the plasma 11 ignited in the discharge gap 9. Through this approach, by high-energy conversions of energy at electron temperatures of 4-6 eV, high photon densities >>10\hat{ }15/s with wavelengths in the UVC range occur, as can be seen in FIG. 2.

With the UVC wavelengths here high reflection rates of up to 97% arise through the dielectric used and the structure of the electrodes, which cannot be achieved in known assemblies according to the prior art.

By contrast with the use of UVC radiation sources used available on the market, 1,000 to 10,000 times higher photon densities are generated.

In order to make a comparison of a 20 W situation between UVC lamps and the electrode from the present plasma filter unit, 6 UV-C lamps of 20 cm in length are need, which irradiate a cross section through a ventilation shaft 21 transparent to UV-C through which air flows, which in this case is also enclosed by an aluminum tube 21 with a high degree of reflection for UV radiation of the selected wavelength.

The aerosols contained in the air flow pass through the UVC activation area over 20 cm in appr. 0.11 s with a 100 to 1000 times lower photon density compared to the serpentine gap of the electrode facility 2, where the aerosols can stay for minutes due to the particle trap. The inactivation rate therefore lies by the product of density x time many orders of magnitude higher than with conventional UV-C lamps. By connection of further electrode facilities 2 behind one another the effectiveness of the plasma filter facility 1 can be increased even further.

Described in more detail below is the said particle trap effect for inactivation of the aerosols. Generally aerosols consist of solid or liquid particles of different sizes. A large part are those in the 100 μm ranges. The starting point can however also be a proportion of much smaller particles, smaller than 5 μm or smaller than 2.5 μm: DGUV Rule 102-001, September 2019 Edition, re. “Regeln für Sicherheit and Gesundheit bei Tätigkeiten mit Biostoffen im Unterricht” (Rules for Safety during Activities with Biological Agents in the Classroom) (of the Deutschen Gesetzlichen Unfallversicherung e.V. (German Social Accident Insurance) DGUV. While droplets of 100 μm in diameter need around 6 seconds to fall to the ground from a height of 2 m, droplets of 10 μm diameter need 10 minutes for the same distance, droplets of 1 μm need 16.6 hours: Source Kappstein, Ines. Nosokomiale Infektionen: Prävention, Labordiagnostik, antimikrobielle Therapie; 122 Tabellen (. Deutschland, Thieme, 2009 (Nosocomial Infections: Prevention, Laboratory Diagnostics, Anti-microbial Therapy; 122 Tables). Smaller particles therefore stay longer in the air, can be distributed in the room via air movement and influence the incidence of infection.

The effect of the electrode design and the arrangement of the electrode facility 2 is that above all the aerosol particle fraction definitively involved in the occurrence of infections stay between the composite electrodes 3, 4 and can be effectively decontaminated there. Best HEPA filters only exclude particle sizes up to 0.3 μm.

As can be seen from FIG. 3, FIG. 7 and FIG. 8, a very large proportion of the so-called suspended aerosol particles classified as dangerous stay in the columnar serpentine structure of the electrode facility 2, where the UV-C radiation is generated. The proportion of the suspended aerosol particles can in this way be inactivated by the effect of the UVC beams. Thus even aerosols with particle sizes through dose powers arising below 0.3 μm are inactivated.

Due to the high photon densities and the enforced dwell time of defined particle fractions high inactivation/decontamination rates of are obtained.

It is assumed that a superspreader generates up to 200,000 2×10\hat{ }5 Sars-COV-2 virus particles per second and cubic meter source: Michael Riediker; Dai-Hua Tsai, Estimation of Viral Aerosol Emissions From Simulated Individuals with Asymptomatic to Moderate Coronavirus Disease 2019, JAMA Network doi:10.1001/jamanetworkopen.2020.13807. In a closed space of for example 50 m\hat{ }3 volume, in this way almost 7×10\hat{ }5-7×10\hat{ }7 virus particles per cubic meter can accumulate from one person. Due to the high number of photons generated in the present plasma filter facility 1, with far more than 1015 photons/s, each particle is subjected to a high number of UVC quanta hits. Due to the very long dwell times of the particles in the serpentine discharge gaps 9 of the electrode facility 2 an efficient inactivation is made possible. These results can lead to a high level of effectiveness and efficiency of the present plasma filter unit

The design of the plasma filter facility 1 has advantages compared to conventional solutions. Due to the overall design of the present plasma filter facility 1 laminar air flows with very little loss of pressure of around 5-6 Pa per electrode facility 2, complete design appr. 50 Pa are achievable.

Through a provision of application-specific filter sizes a very compact and small design of just a few cm, and also a scalability for large filter systems in the meter range is possible, as can be seen in FIG. 7.

The plasma filter facility 1 can make high throughflow rates of 250-300 m³/h with laminar flow conditions.

Due to the compact design and standard materials the costs of manufacturing are low. A low profile made possible by the shape of the plasma filter facility 1 allows a direct integration of the plasma filter facility 1 in for example ceiling ventilators. An integration of the plasma filter facility 1 into automotive and mobility applications is made possible due to the compact design and a reduced noise emission through the laminar air flow.

The present plasma filter facility 1 can replace existing filter systems in aircraft. Due to the saving in weight and installation space, new design options with regard to the cabin and supporting structures are produced. A direct substitution of complex filter cascades in buildings is possible through the plasma filter facility 1.

This makes a reduced used of HEPA filter units of different variants possible, an improvement of the energy balance, a reduction of waste and also a lengthening and/or change of maintenance intervals. Due to the compact design it is possible explicitly to screen the plasma filter facility 1 to make possible a high level of electromagnetic compatibility by arrangements, see HF and B field compatibility.

Due to the high electromagnetic sensitivity of electronic and imaging components, such as for example magnetic resonance tomography devices, particular importance must be attached to the electromagnetic compatibility of the plasma units and the power supply technology. Existing solutions and solutions available on the market would have to be expensively adapted with respect to plasma generator and/or power electronics as well as to the electrode technology used. In most cases an adaptation is not possible.

In order to guarantee a fully screened EMC operation, in the present plasma filter facility 1 a structure of three crossed or uncrossed coated electrode facilities 2 is recommended, which are each separated by a spacer frame. This produces a Faraday cage, which fully screens a basic frequency of 10 kHz up to the relative distance 15 between the electrode pins of 1-2 mm. Due to the compact design of the present electrode facility 2 a housing along with power electronics and/or plasma generator in for example a stainless steel tube 21 is possible. A sufficient screening is provided by this.

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element (s) or feature (s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of the embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing embodiments. The present invention may, however, be embodied in many alternate forms and should not be construed as limited to only the embodiments set forth herein.

What is claimed is:

1. A plasma filter device, comprising:
at least one electrode device having a first composite electrode configured in a planar manner and a second composite electrode configured in a planar manner, wherein
the first composite electrode and the second composite electrode are arranged coplanar relative to one another in a main surface plane of the at least one electrode device, and the first composite electrode and the second composite electrode are spatially separated from one another by a discharge gap, and
each of the first composite electrode and the second composite electrode has a respective electrode sheet that, at least on a boundary surface of the respective electrode sheet to the discharge gap, has a respective dielectric coating; and
a power source configured to provide an AC voltage to the at least one electrode device, the AC voltage being parameterized to bring about a formation of a plasma through a dielectric barrier discharge in the discharge gap, wherein
the plasma filter device is configured to guide a gas along a main direction of flow, the main direction of flow being aligned in parallel with a normal of the main surface plane of the at least one electrode device, through the discharge gap.

2. The plasma filter device as claimed in claim 1, wherein
each of the first composite electrode and the second composite electrode has a comb structure, which has electrode fingers, and
comb structures of the first composite electrode and the second composite electrode engage with each other and are separated from one another by the discharge gap.

3. The plasma filter device as claimed in claim 1, wherein the respective electrode sheet of each of the first composite electrode and the second composite electrode includes at least one of aluminum or an aluminum alloy.

4. The plasma filter device as claimed in claim 1, wherein the respective electrode sheet of each of the first composite electrode and the second composite electrode includes non-corrosive steel.

5. The plasma filter device as claimed in claim 1, wherein the respective dielectric coating of each of the first composite electrode and the second composite electrode includes one or more polymers.

6. The plasma filter device as claimed in claim 5, wherein the respective dielectric coating of each of the first composite electrode and the second composite electrode includes one or more fluoroplastics.

7. The plasma filter device as claimed in claim 6, wherein the respective dielectric coating of each of the first composite electrode and the second composite electrode includes polyvinylidene difluoride.

8. The plasma filter device as claimed in claim 6, wherein the respective dielectric coating of each of the first composite electrode and the second composite electrode features polytetrafluorethylene.

9. The plasma filter device as claimed in claim 1, wherein the respective dielectric coating of each of the first composite electrode and the second composite electrode includes graphite fluoride.

10. The plasma filter device as claimed in claim 1, wherein the respective dielectric coating of each of the first composite electrode and the second composite electrode includes one or more ceramics.

11. The plasma filter device as claimed in claim 10, wherein the respective dielectric coating of each of the first composite electrode and the second composite electrode includes barium titanate.

12. The plasma filter device as claimed in claim 10, wherein the respective dielectric coating of each of the first composite electrode and the second composite electrode includes kaolinite.

13. The plasma filter device as claimed in claim 10, wherein the respective dielectric coating of each of the first composite electrode and the second composite electrode includes a blend, including proportions of at least one of kaolinite, aluminum oxide, titanium oxide, chromium oxide, barium titanate or other ceramic powders.

14. The plasma filter device as claimed claim 1, wherein
the plasma filter device includes at least two electrode devices, and
the plasma filter device includes a holder device configured to arrange the at least two electrode devices in an electrode arrangement, wherein the at least two electrode devices are aligned in parallel with one another, and along the main direction of flow behind one another, and separated from one another in pairs by a distance.

15. The plasma filter device as claimed in claim 14, wherein neighboring electrode devices of the at least two electrode devices are aligned rotated by 90 degrees relative to one another around the main direction of flow.

16. The plasma filter device as claimed in claim 1, further comprising:
at least one dust filter arranged in the main direction of flow before the at least one electrode device.

17. The plasma filter device as claimed in claim 1, further comprising:
at least one active carbon filter arranged in the main direction of flow after the at least one electrode device.

18. An electrode device for a plasma filter device, the electrode device comprising:
a first composite electrode configured in a planar manner; and
a second composite electrode configured in a planar manner, wherein
the first composite electrode and the second composite electrode are arranged coplanar to one another in a main surface plane of the electrode device, and the first composite electrode and the second composite electrode are spatially separated from one another by a discharge gap, and
each of the first composite electrode and the second composite electrode has a respective electrode sheet that, at least on a boundary surface of the respective electrode sheet to the discharge gap, has a respective dielectric coating.

19. A method for operating a plasma filter device, the plasma filter device including at least one electrode device, wherein the at least one electrode device includes a first composite electrode configured in a planar manner and a second composite electrode configured in a planar manner, wherein the first composite electrode and the second composite electrode are arranged coplanar to one another in a main surface plane of the at least one electrode device, and the first composite electrode and the second composite electrode are spatially separated from one another by a discharge gap, and wherein each of the first composite electrode and the second composite electrode has a respective electrode sheet that, at least on a boundary surface of the respective electrode sheet to the discharge gap, has a respective dielectric coating, wherein the method comprises:
providing, through a power source of the plasma filter device, an AC voltage to the at least one electrode device such that a plasma is generated by a dielectric barrier discharge in the discharge gap; and
routing, through the plasma filter device, a gas along a main direction of flow, which is aligned in parallel with a normal of the main surface plane of the at least one electrode device, through the discharge gap.

20. The plasma filter device as claimed claim 2, wherein
the plasma filter device includes at least two electrode devices, and
the plasma filter device includes a holder device configured to arrange the at least two electrode devices in an electrode arrangement, wherein the at least two electrode devices are aligned in parallel with one another, and along the main direction of flow behind one another, and separated from one another in pairs by a distance.

21. The plasma filter device as claimed in claim 20, wherein neighboring electrode devices of the at least two electrode devices are aligned rotated by 90 degrees relative to one another around the main direction of flow.

22. The plasma filter device as claimed in claim 20, further comprising:
at least one dust filter arranged in the main direction of flow before the at least one electrode device.

23. The plasma filter device as claimed in claim 20, further comprising:
at least one active carbon filter arranged in the main direction of flow after the at least one electrode device.

24. The plasma filter device as claimed in claim 2, further comprising:
at least one dust filter arranged in the main direction of flow before the at least one electrode device.

25. The plasma filter device as claimed in claim 2, further comprising:
at least one active carbon filter arranged in the main direction of flow after the at least one electrode device.
